(19) United States
(12) Patent Application Publication
(10) Pub. No.: US 2006/0173232 A1
(43) Pub. Date: Aug. 3, 2006

(54) HDR adapter for electronic radiation source applicator

(76) Inventors: Paul A. Lovoi, Saratoga, CA (US); Alex Lim, Santa Clara, CA (US)

Correspondence Address: Thomas M. Freiburger, P.O. Box 1026, Tiburon, CA, 94920, US

(21) Appl. No.: 11/323,347

(22) Filed: Dec. 30, 2005

Related U.S. Application Data
(63) Continuation-in-part of application No. 10/464,140, filed on Jun. 18, 2003.

(51) Int. Cl. A61N 5/00

(52) U.S. Cl. 600/1

(57) ABSTRACT

An applicator designed for use with a miniature electronic x-ray source, having a relatively large guide for the electronic source, is modified by an adapter inserted into the source guide to produce a narrower-diameter guide that will receive a standard afterloader that irradiates with isotopes.

This application is a continuation-in-part of application Ser. No. 10/464,140 filed Jun. 18, 2003, which is incorporated by reference herein in its entirety.

### BACKGROUND OF THE INVENTION

This invention concerns therapeutic radiation treatment, and especially apparatus for brachytherapy radiation treatment.

Electronic radiation sources are disclosed in U.S. Pat. No. 6,319,188 as well as in co-pending applications Ser. No. 10/464,140 filed Jun. 18, 2003, Ser. No. 10/371,401 filed Feb. 21, 2003 and Ser. No. 10/397,498 filed Mar. 26, 2003, of Xoft, Inc. These electronic x-ray sources range from about 3 or 4 mm in diameter down to about 1 mm in diameter. Applicators for electronic sources have been proposed, particularly for post-operative brachytherapy of a breast tumor resection cavity, and also for-other tissue treatment. Such an applicator has a shaft with a lumen or guide that receives a catheter device with the electronic x-ray source at or near its tip. This is connected by a high voltage cable back to a controller operated by the surgeon or technician.

It is desirable to adapt the applicator designed for electronic sources to use with standard afterloaders, in order to provide the versatility of only one type of applicator in a treatment facility, useable with both electronic and isotope x-ray sources.

### SUMMARY OF THE INVENTION

In accordance with the current invention, an applicator designed for use with a miniature electronic x-ray source, and having a relatively large channel or guide for the electronic source, is modified by an adapter inserted into the source guide to produce a narrower-diameter guide that will receive a standard afterloader for irradiation with isotopes.

### DESCRIPTION OF THE DRAWINGS

FIG. 1 is an exploded-type view showing, schematically, an applicator intended for use with an electronic x-ray source, having a relatively large internal guide, and an adapter device for insertion into the applicator, to produce a smaller guide lumen that will receive an afterloader carrying an HDR seed or pellet.

FIG. 2 is a cross section view through the adapter device, showing features of a preferred embodiment.

### DESCRIPTION OF PREFERRED EMBODIMENTS

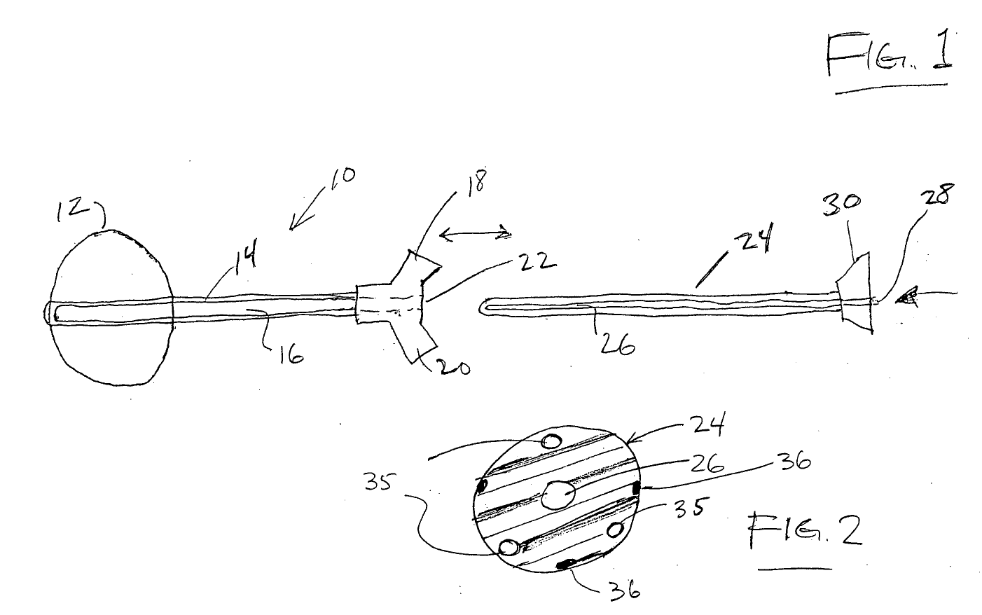

In the drawing, FIG. 1 shows a balloon applicator 10 designed for use with an electronic x-ray source. The applicator 10 includes a balloon 12 and a shaft 14 within which is a central guide or lumen 16 to receive a catheter or probe carrying an electronic radiation source, not shown. Other lumens are included in the shaft 14, for various purposes, but these are not shown in the drawing. At the proximal end of the applicator are ports 18 and 20 for cooling, suction, and/or other purposes, as well as the central port 22 to receive the radiation source.

In one typical applicator of Xoft, Inc., the balloon applicator 10 has a service centering lumen 16 with a diameter of about 4 to 6 mm, for breast therapy or other radiation therapy application.

FIG. 1 shows an adapter 24 according to the invention, for insertion through the center port 22 into the service centering lumen or guide 16 of the applicator. The HDR adapter 24 has just sufficient clearance for entry into the service lumen 16, and has a smaller center lumen 26, with a proximal entry 28, the diameter of this lumen 26 being about 1 mm for an HDR pellet to be inserted, via a standard afterloader (not shown).

The adapter 24, which serves to provide a smaller service lumen in the applicator 10, can be formed of injection molded plastic such as Hytrel, silicone or other biocompatible material. At the proximal end of the adapter is an end piece 30 which may be attached to the elongated adapter shaft 24 or which could be integrally formed by injection molding. This proximal end-piece 30 is configured to nest with the proximal end of the existing applicator device 10. Preferably some form of positive lock or positive fix is included, such as a snap-in or a thread. There should be a positive visual (and also, optimally, tactile) indication that the adapter is in the right place so that treatment is correct.

For this purpose FIG. 2 shows one form of positive position locator, with the adapter shaft 24 shown in cross section. Outer lumens 35 are provided in the adapter shaft 24, forward of flexible material such as silicone. Three are shown, spaced equilaterally, but more can be provided. Fluid pressure is applied to these lumens 35 after the adapter is inserted fully into the guide 16 of the adapter 10. This expands the adapter shaft outwardly at the locations of the pressure-receiving lumens 35 to secure the adapter against the inner lumen or guide wall 16 of the balloon applicator 10. The expansion of the adapter shaft also acts to center the adapter 24, while securing it in place against axial movement.

It is also possible, via use of the HDR adapter 24, to provide verification of dose using dosimeters 36 (three shown in FIG. 2) that are on the outer wall of or embedded in the outer surface of the adapter shaft 24. These can be wirelessly connected to a treatment point existent to feed back, in real time, dose received at the locations of the dosimeters, thus enabling by extrapolation calculation of the dose delivered at the tissue. This can provide not only verification that treatment was according to plan, but real-time feedback control of the treatment procedure itself, via the treatment planning system. With an HDR source pullback rate or dwell time at each of a series of stepped locations can be changed in real time based on the feedback.

Note also that the feedback control dosimeters 36 can be located in the applicator shaft 14 of the applicator 10, or in the balloon 12 of the applicator.

The above described preferred embodiments are intended to illustrate the principles of the invention, but not to limit its scope. Other embodiments and variations to these preferred embodiments will be apparent to those skilled in the art and may be made without departing from the spirit and scope of the invention.

1. In combination with a brachytherapy applicator having an internal guide sized to receive an electronic x-ray source, an adapter for the applicator comprising a generally tubular sleeve or shaft having an outer diameter sized to fit closely within the guide of the applicator and an inside diameter sized to receive an afterloader carrying an isotope radiation source.

2. The combination as in claim 1, including position fixing means for holding the adapter firmly in place within the internal guide of the applicator.

3. The combination as in claim 2, wherein the position fixing means comprises a plurality of expandable lumens in the adapter sleeve or shaft near an exterior surface, the adapter sleeve or shaft being flexible, such that fluid under pressure can be admitted to the expandable lumens to expand the outer surface of the adapter into firm contact with the internal guide of the applicator.

4. The combination as in claim 1, wherein the sleeve or shaft of the adapter includes at least one dosimeter capable of wirelessly communicating with a treatment planning system, for verification of dose delivered in a brachytherapy treatment using the applicator.
